(19) United States
(12) Patent Application Publication
(10) Pub. No.: US 2002/0087204 A1
(43) Pub. Date: Jul. 4, 2002

(54) Flexible transcutaneous energy transfer (TET) primary coil

(76) Inventors: Robert T. V. Kung, Andover, MA (US); Robert M. Hart, Arlington, MA (US); Farhad Zarinetchi, Chelmsford, MA (US)

Correspondence Address: NUTTER MCCLENNEN & FISH LLP, ONE INTERNATIONAL PLACE, BOSTON, MA, 02110, US

(21) Appl. No.: 09/754,835

(22) Filed: Jan. 4, 2001

(51) Int. Cl. A61N 1/08

(52) U.S. Cl. 607/61

(57) ABSTRACT

A substantially flexible primary coil for use in a transcutaneous energy transfer (TET) device. When in use, the primary coil conforms to the contours of a patient's body, and, more particularly, to the shape of the skin under which a secondary coil of the TET is implanted. The shape of the primary coil adjusts with short term and long term changes in the contour of the patient's skin, enabling the primary coil to maintain a desired relative position with the implanted secondary coil as the patient changes position, posture or orientation, as well as over time as the patient loses or gains weight. The primary coil includes a shape-retaining base ring defining a primary plane. The base ring is firm, yet is able to conform to the contours of the substrate upon which it is placed. Attached to the base ring is a conducting element such as flexible wire which is coiled to form a plurality of nearly concentric windings. The windings are able to move at any angle, including transverse, to the primary plane so as to adopt the shape of the skin against which it is placed. In this way, the primary coil can be placed on any irregular surface and take the shape of the irregularities. A binding element is bound to the plurality of windings so that the near concentricity of the windings is maintained when the windings move.

### CROSS-REFERENCE TO RELATED APPLICATIONS

This application is related to commonly-owned U.S. patent application Ser. No. 09/110,608, filed Jul. 6, 1998 entitled “Magnetic Shield for Primary Coil of Transcutaneous Energy Transfer Device”, now currently pending, and U.S. patent application Ser. No. 09/347,322, filed Jul. 2, 1999 entitled “Magnetic Shield for Primary Coil of Transcutaneous Energy Transfer Device”, also now currently pending, both of which are hereby incorporated by reference herein and elsewhere in this application.

### FIELD OF THE INVENTION

This invention relates to transcutaneous energy transfer (TET) devices and, more particularly, to a flexible primary coil for such a device.

### BACKGROUND OF THE INVENTION

Many medical devices are now designed to be implantable, including pacemakers, defibrillators, circulatory assist devices, cardiac replacement devices, cochlear implants, neuromusculator stimulators, biosensors, and the like. Since almost all active devices (devices that perform work) and many passive devices (devices that do not perform work) require a source of power, inductively coupled transcutaneous energy transfer (TET) devices and information transmission systems for such devices are coming into increasing use. A TET system may be employed to supplement, replace, or charge an implanted power source, such as a rechargeable battery. Unlike other types of power transfer systems, TET systems provide power to the implanted electrical and/or mechanical device, or recharge an internal power source, without use of a percutaneous lead. As a result, TET systems reduce the risk of infection and increase patient comfort and convenience.

Generally, TET systems include a transcutaneous transformer having an external primary coil operationally aligned with an implanted secondary coil. A primary circuit drives the primary coil to induce alternating current in the subcutaneous secondary coil, typically for transformation to direct current to power the implanted device or power source. The non-implanted portions of conventional TET systems, including the primary coil and its drive circuitry, are attached externally to the patient, typically by a belt or other fastener or garment. Implantable medical devices must be carefully designed with respect to both size and shape in order to minimize the risk of necrosis. This is particularly true for medical devices which are implanted in subcutaneous tissue. The secondary coil of a TET, for example, is typically implanted between the dermis layer of the skin and the subcutaneous tissue. Accordingly, it has generally been recognized that the size of implanted devices should be as small as possible consistent with the functional integrity of the implanted device. In addition to designing implantable devices with a minimal volume, intuitive considerations have led designers to avoid sharp corners. Conventional secondary coils, for example, typically reside in a housing with a truncated cylindrical shape with rounded corners, mimicking a hockey puck. When implanted, the secondary coil housing can form a pronounced bulge, or protrusion, in the patient's skin.

In an attempt to provide effective coupling of the coils, some conventional primary coils are formed having a rigid, truncated conical shape that is intended to correspond to the shape of the bulge in the patient's skin formed by the secondary coil housing. Other conventional primary coils take a planar ring shape with the center of the ring being open to receive the protruding secondary coil housing. One common drawback to such primary coils is that they often do not seat comfortably over the irregularly shaped skin, causing the patient to adjust consciously or unconsciously the primary coil. Furthermore, despite the shape of the primary coil, a patient's shifting position can change the shape of the secondary coil protrusion. Because conventional primary coils rely on the protrusion for maintaining coil alignment, such a change in shape facilitates the undesired movement of the primary coil. Such events can cause the coils to decouple, which, in turn can cause the implanted medical device to lose power or switch to an implanted battery for its power source. Repetition of such occurrences can reduce the life of the implanted battery, if any, or cause a loss of the functions provided by the implanted medical device an implanted battery is not provided or operational.

### SUMMARY OF THE INVENTION

The present invention is directed to a substantially flexible primary coil for use in a transcutaneous energy transfer (TET) device. When in use, the primary coil is able to conform to the contours of a patient's body, and, more particularly, to the shape of the skin under which a secondary coil of the TET is implanted. Such a primary coil significantly enhances the comfort of a patient receiving the TET device. Not only does the primary coil provide an increased quality of life, it also reduces the likelihood that the patient will adjust the primary coil in an attempt to alleviate discomfort. Furthermore, the shape of the primary coil adjusts with short term and long term changes in the contour of the patient's skin. This enables the primary coil to maintain a desired relative position with respect to the implanted secondary coil as the patient changes position, posture or orientation, or even as the patient changes shape, such as when the patient loses or gains weight. Thus, the primary coil provides a more effective transfer of energy to the implanted secondary coil without significant energy loss due to improper coupling of the coils.

A number of aspects of the invention are summarized below, along with different embodiments that may be implemented for each of the summarized aspects. It should be understood that the embodiments are not necessarily inclusive or exclusive of each other and may be combined in any manner that is non-conflicting and otherwise possible. It should also be understood that these summarized aspects of the invention are exemplary only and are considered to be non-limiting.

In accordance with one aspect of the invention, a flexible transcutaneous energy transfer primary coil is provided. The primary coil includes a substantially shape-retaining base ring defining a primary plane, a conducting element in the form of a plurality of substantially concentric windings attached to the base ring, and at least one binding element bound to a plurality of the windings to substantially maintain the concentricity of the bound windings while allowing the bound windings to move in a direction transverse to the primary plane with respect to other bound windings, thereby enabling the conducting element to conform to a shape of a patient's skin or other surface.

In another aspect of the invention, a coil for transferring electrical power to a subcutaneous utilization device is disclosed. The coil is formed from a conducting element and has a series of windings. The windings are attached to one another by at least one binding element. The coil also includes a substantially shape-retaining base ring that is connected to the binding element and surrounds the conducting element. The binding element and conducting element are constructed and arranged such that each winding is translatable to a location not in a plane defined by the base ring and non-translated windings.

In accordance with a still further aspect of the invention, a transcutaneous energy transfer device is provided. The device includes a flexible external primary coil to which energy to be transferred is applied, and an implanted secondary coil adapted to be inductively coupled to the primary coil and to apply energy to an implanted utilization device. The primary coil also includes a substantially shape-retaining base ring defining a primary plane. The base ring is firm, yet is able to conform to the contours of the skin. Attached to the base ring by at least one binding element is a conducting element such as flexible wire which is coiled to form a plurality of substantially concentric windings. The windings are able to move at any angle, including transverse, to the primary plane so as to conform to the shape of the skin against which it is placed. In this way, the primary coil can conform to the irregular surface and take the shape of the irregularities. At least one binding element is bound to the plurality of windings so that the near concentricity of the windings is maintained when the windings move.

In a further aspect of the invention, an external primary coil for use in a transcutaneous energy transfer system having an implantable secondary coil configured to be inductively coupled to the external primary coil to deliver energy received from the primary coil to an implanted device is provided. The primary coil is flexible, taking on a shape in response to external forces such that when applied to a patient's skin under which the secondary coil is implanted, the primary coil continually conforms to the patient's skin over time.

In another aspect of the invention, a transcutaneous energy transfer system is provided. The TET system includes a flexible external primary coil to which energy to be transferred is applied; and a secondary coil coupled to the primary coil, the secondary coil being implanted to be connected to apply energy to a subcutaneous utilization device. The primary coil is formed from a conducting element and having a series of windings, each winding being able to flex with respect to an adjacent winding, wherein the windings are attached to one another by at least one binding element.

In a further aspect of the invention an external primary coil for use in a transcutaneous energy transfer system having an implantable secondary coil configured to be inductively coupled to the external primary coil to deliver energy received from the primary coil to an implanted device is provided. The individual windings of the primary coil can translate relative to each other to enable the primary coil to conform continually to a patient's skin under which the secondary coil is implanted.

In one embodiment, the binding element comprises a plurality of threads. The threads are bound at one end to the base ring, and are wound around the flexible wire to form an interconnected network which allows the wire to flex and move so as to conform to the shape of the skin surface against which it is placed. Preferably, the threads are formed from an elastic material to facilitate the flexion of the coil. At the same time, the threads are sufficiently durable to maintain the near concentric arrangement of the windings.

In another embodiment, the binding element comprises a flexible substrate. This substrate could be fabric, such as spandex or mesh fabric. The fabric may be bound to the base ring and to the windings by means of stitches. The fabric can be pleated in an accordion pattern so that the wire can move transverse to the base ring, and adapt to the shape of the bulge, or protrusion, on the skin surface where the secondary coil has been implanted. In addition, the fabric can be porous to allow for ventilation to the skin to improve the wearer's comfort.

In yet another embodiment, more than one type of binding element can be utilized in the present invention. A combination of threads and fabric can be used to bind the windings in a near concentric arrangement, while allowing the wire to flex and move transverse to the base ring.

In yet a further embodiment, the primary coil can function without the need for the shape-retaining base ring. It is contemplated in this embodiment that the primary coil, which includes the conducting element and the binding element, is able to substantially maintain the shape of the substrate upon which it is placed, without the need for the base ring.

In certain embodiments, the primary coil can also include a flexible magnetic shield and/or a compliant skin-compatible cushion such as those described in U.S. patent application Ser. Nos. 09/110,608 and 09/347,322, both of which are assigned to the assignee of the present invention, and which are herein incorporated by reference.

For example, an unshielded primary coil generates an alternating magnetic field which is directed in substantially equal parts in two directions: one direction is toward the secondary coil, where it induces a current in a secondary coil located in the field, and the second direction is away from the secondary coil where the magnetic field energy does not induce a current in the secondary coil. If a higher percentage of the magnetic field from the primary coil could be directed to the implanted secondary coil, the energy required to drive the TET device could be reduced. Thus, it is contemplated that a flexible magnetic shield can be placed on top of the flexible primary coil, on the surface facing away from the patient, to direct a larger portion of the magnetic field toward the secondary coil. The magnetic shield can serve to enhance the efficiency of energy transfer across the skin boundary by the TET device.

Various aspects of the present invention and embodiments thereof provide certain advantages and overcome certain drawbacks of conventional techniques. Not all aspects and embodiments share the same advantages and those that do may not share them under all circumstances. This being said, the primary coil of the present invention provides the significant advantage of being able to adjust shape to maintain conformity to the contours of a patient's body, significantly enhancing patient comfort and TET performance. Further features and advantages of the present invention as well as the structure and operation of various embodiments of the present invention are described in detail below with reference to the accompanying drawings. In the drawings, like reference numerals indicate like or functionally similar elements.

### BRIEF DESCRIPTION OF THE DRAWINGS

This invention is pointed out with particularity in the appended claims. The above and further features and advantages of this invention may be better understood by referring to the following description when taken in conjunction with the accompanying drawings, in which:

FIG. 1 is a diagrammatic view of a patient with an implanted medical device and a flexible TET primary coil in accordance with the teachings of this invention;

FIG. 2 is a side cutaway view of the configuration of the primary and secondary coils of a TET coil pair in accordance with the teachings of this invention;

FIG. 2A is an exploded side view of the configuration of the primary and secondary coils of another TET coil pair;

FIG. 3 is a top view of a flexible TET primary coil for one embodiment of the invention;

FIG. 3A is an elevated side view of the flexible TET primary coil of FIG. 3 on the patient's skin;

FIG. 4 is a top view of a flexible TET primary coil for another embodiment of the invention;

FIG. 4A is an elevated side view of the flexible TET primary coil of FIG. 4 on the patient's skin;

FIG. 5 is a top view of a flexible TET primary coil for yet another embodiment of the invention; and

FIG. 6 is a side cutaway view of an exemplary magnetic shield in use with a TET coil pair.

### DETAILED DESCRIPTION OF THE INVENTION

The present invention is directed to a substantially flexible primary coil for use in a transcutaneous energy transfer (TET) device. When in use, the primary coil is able to conform to the contours of a patient's body, and, more particularly, to the shape of the skin under which a secondary coil of the TET is implanted. Such a primary coil significantly enhances the comfort of a patient receiving the TET device. Not only does the primary coil provide an increased quality of life, it also reduces the likelihood that the patient will adjust the primary coil in an attempt to alleviate discomfort. Furthermore, the shape of the primary coil adjusts with short term and long term changes in the contour of the patient's skin. This enables the primary coil to maintain a desired relative position with the implanted secondary coil as the patient changes position, posture or orientation, as well as over time as the patient loses or gains weight. Thus, the primary coil provides a more effective transfer of energy to the implanted secondary coil without significant energy loss due to improper coupling of the coils.

In the exemplary implementation disclosed herein, an electrohydraulic implantable replacement heart is the implanted utilization device. The replacement heart receives power directly from the TET device or from an implantable battery that also receives power from the TET device. It should be understood, however, that the primary coil and TET device of the present invention can be implemented with any implantable device now or later developed. This includes other cardiac replacement devices, devices that assist one or both ventricles of the heart, pacemakers, defibrillators, cochlear implants, neuromusculator stimulators, biosensors, and other active and passive implantable devices.

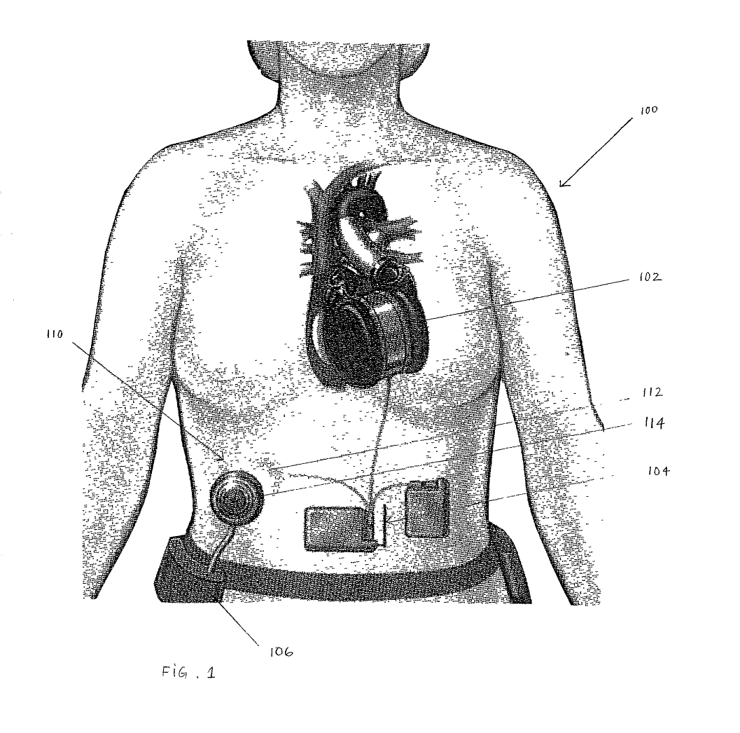

FIG. 1 shows an exemplary medical system 100 utilizing a transcutaneous energy transfer (TET) coil pair 110 including the primary coil 114 of the present invention. As illustrated, a patient has an implanted cardiac replacement device 102 connected by way of controller 104 to a secondary coil 112, both of which are also implanted inside the patient. External to the patient is a primary coil 114 which is connected to an AC power source (not shown). The primary coil 114 pairs with the secondary coil 112 for effecting energy transfer. When the primary coil 114 is placed against the patient's skin 120 and over the secondary coil 112, the paired coils 110 serve as a transformer, inducing AC current in the secondary coil 112. The AC current is passed through a converter (not shown) in the controller 106 which converts the AC current to DC to power the cardiac assist device 102. As noted, while the illustrated embodiment depicts the transcutaneous energy transfer system 100 of the invention deployed with an implanted cardiac replacement device 102, the invention can be used with any implantable device requiring energy.

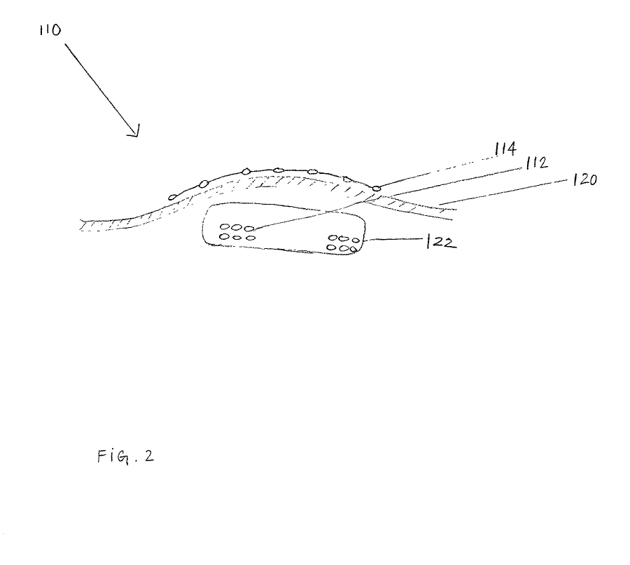

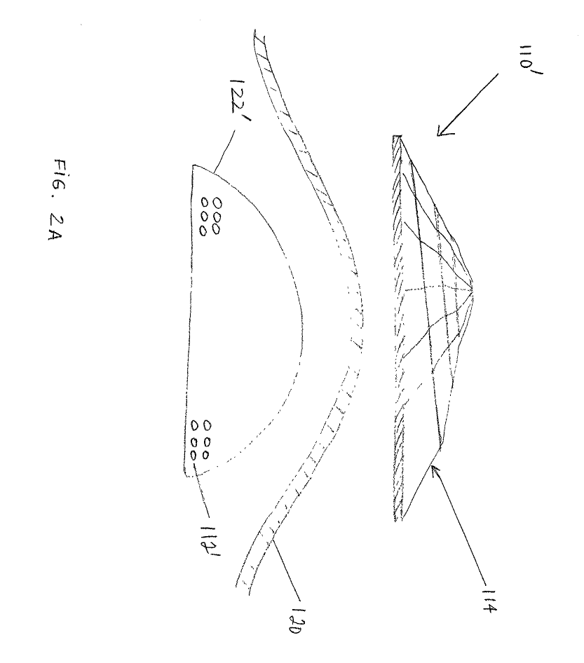

FIGS. 2 and 2A illustrate two exemplary TET pair systems 110, 110′ where secondary coil 112, 112′ of the pair can create a non-planar, or irregular, skin surface when implanted. In FIG. 2, a cylindrically shaped housing 122 encasing a planar secondary coil 112 results in a bulge under the patient's skin 120 when implanted. The irregularities are especially pronounced when a conical secondary coil 112′ having dome-shaped housing 122′ is utilized, as illustrated in FIG. 2A. In both instances, the primary coil 114 of the present invention is constructed such that the primary coil 114 is able to take the shape of the bulge, or protrusion, when placed against the skin 120. Because the primary coil 114 of the invention is able to flex and conform to the shape of the bulge, it can remain inductively coupled to the secondary coil, resulting in a more efficient transfer of energy to the secondary coil 112, 112′ . A flexible primary coil 114 is also more comfortable for the patient. Since the coil 114 is able to conform to the shape of the bulge, the wearer is relieved of having to exert pressure against a rigid primary coil (and hence the patient's skin 120) to force the coil to completely enclose the bulge, and thus the implanted secondary coil 112, 112′. Furthermore, the flexibility of the coil 114 results in less binding and drafting of the patient's skin 120 during use. And, the versatility of the flexible primary coil 114 of the present invention allows it to be used in conjunction with a secondary coil housing 122, 122′ of any shape in a transcutaneous energy transfer system for an implanted medical device.

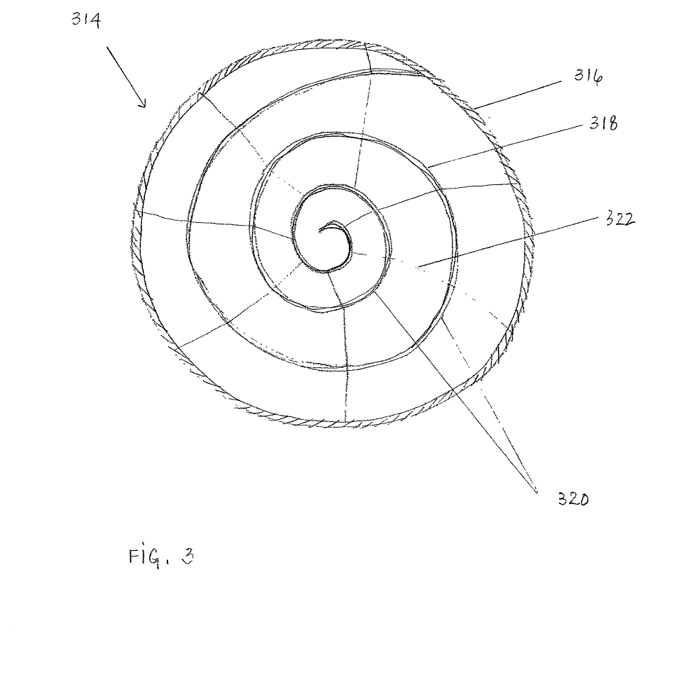

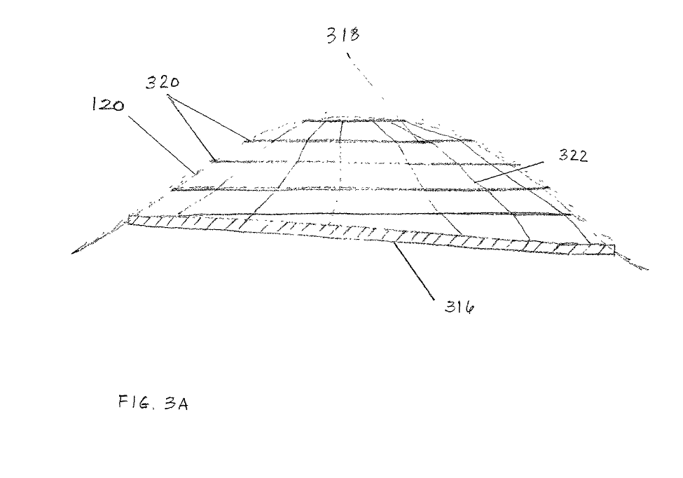

In one embodiment of the present invention, shown in FIG. 3, primary coil 314 includes a substantially shape-retaining base ring 316. The ring 316 can be substantially rigid, or flexible, and can conform to the shape of the substrate against which it is placed. Base ring 316 defines a primary plane from which the flexible wire 318 can move, or translate, at any angle, including transversely to the primary plane. Flexible wire 318 is wound into a plurality of substantially concentric windings 320. A binding element holds the windings 320 together and attaches them to the base ring 316 so that they substantially maintain their concentricity when the wire 318 moves transverse to the primary plane. In this particular embodiment, the binding element is a plurality of threads 322. Each of the threads 322 can be bound to the base ring 316 as well as to a portion of wire 318.

When primary coil 314 is placed against an irregularly shaped skin surface 20, the primary coil 314 takes the shape of the irregular or non-planar surface. As seen in FIG. 3A, the flexible wire 318 is able to move transversely to the base ring 316, wrapping around the bulge of the skin surface 120 created by an implanted secondary coil. The thread 322 allows the windings 320 to move about while still substantially maintaining their concentricity.

The conducting element is formed from a material suitable for transferring energy. More particularly, the conducting element is preferably a flexible wire formed from Litzendraht wire. The flexible wire 318 can comprise a single material, or a plurality of different materials. Additionally, the flexible wire 318 can be single stranded, or can comprise a plurality of strands. The wire 318 can also be braided.

The thread 322 used in the present invention can comprise surgical grade suture thread. The thread 322 can be formed of a polymeric material, such as polyethylene or polyester. Preferably, the thread 322 can be formed from an elastic material to facilitate flexion and conformity of the coil 314 to the skin surface 120, yet is sufficiently strong to hold the wire 318 in near concentric windings 320.

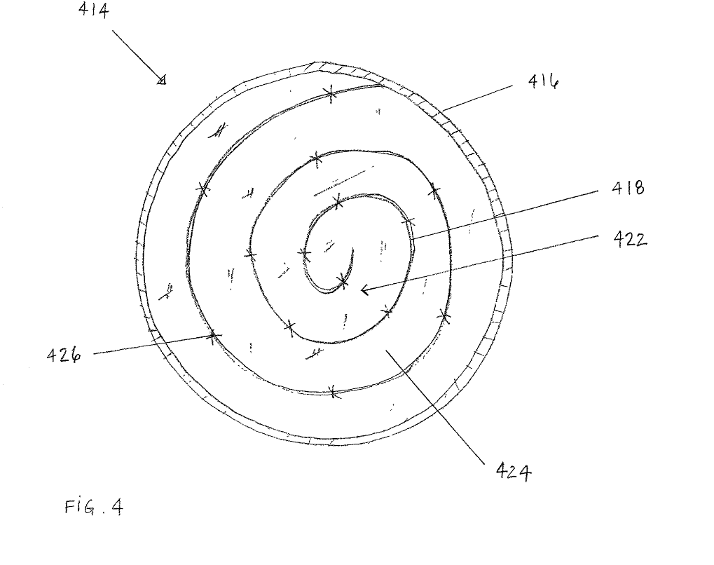

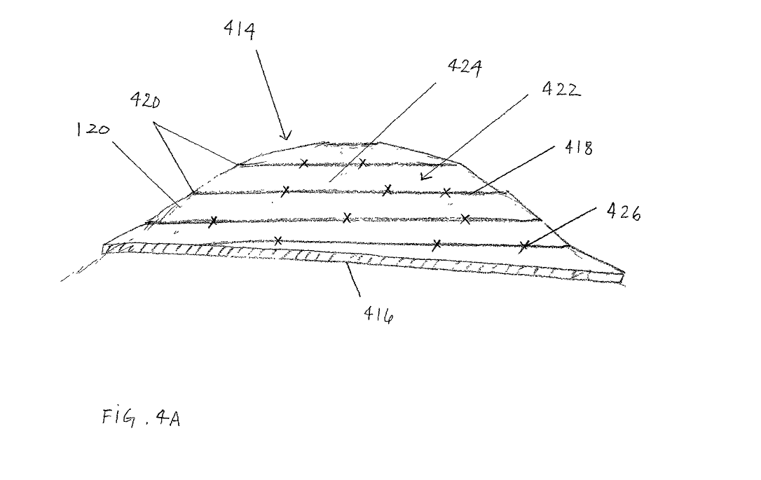

In an alternative embodiment of the present invention shown in FIG. 4, the binding element of the flexible primary coil 414 can comprise a flexible and elastic substrate 422. Flexible primary coil 414 includes a base ring 416 and a flexible wire 418 coiled into a plurality of concentric windings 420 as described for flexible primary coil 314. Flexible primary coil 414 also includes a binding element comprising a fabric 424. The flexible wire 318 can be attached to the fabric 424 with stitches 426.

FIG. 4A shows the flexible primary coil 414 on the outer surface of skin 120. The substrate 422 is sufficiently pliable and elastic such that flexible wire 418 is able to move transversely from the base ring 416, while still maintaining the windings 420 in an approximately concentric arrangement.

The fabric 424 can comprise spandex, or a mesh fabric. Preferably, the fabric 424 is porous so that the patient's skin is able to breathe when the primary coil 414 is in use. This provides comfort to the patient, and prevents excessive perspiration underneath the primary coil 414 which could affect the energy transfer.

Additionally, fabric 424 can be folded in an accordion fashion so as to form pleats (not shown) between consecutive windings 420. The pleats allow free movement of the wire 418 around the skin surface. Further, the pleats prevent uneven folding, or bunching, of fabric in areas between windings 420 lying in close planes.

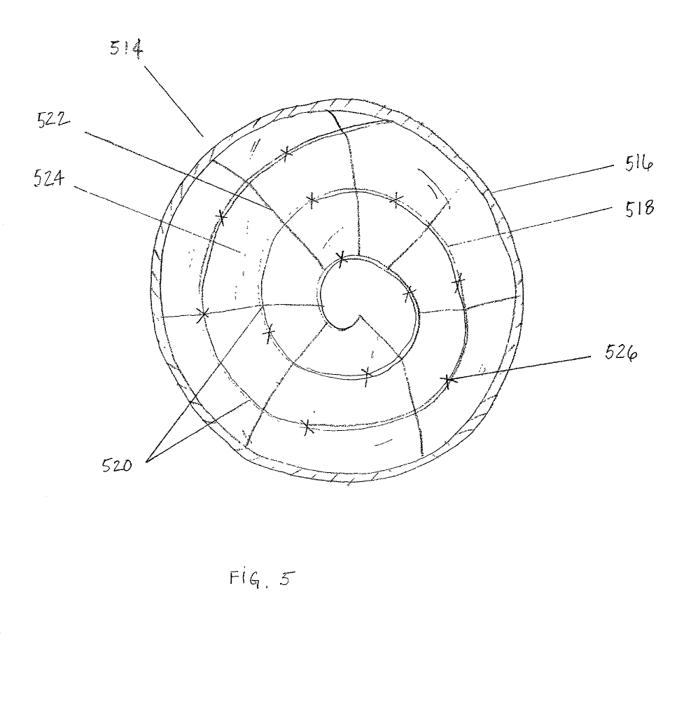

It is contemplated that a combination of different binding elements can be practiced with the instant invention. In yet another embodiment of the present invention, illustrated in FIG. 5, the primary coil 514 can contain both thread 522 and fabric 524 for binding the flexible wire 518. As in the other embodiments described, flexible wire 518 is attached at one end to a substantially shape retaining base ring 516. Thread 522 and fabric 524 are also bound at one end to the base ring 516 as well as to a portion of the wire 518. Additionally, flexible wire 518 is held onto the fabric 524 with stitches 526. Thread 522 and fabric 524 allow the transverse movement of the flexible wire 518 with respect to the base ring 516, while still maintaining the substantial concentricity of the windings 520.

Also, while the disclosed embodiments show binding elements attached to a substantially shape-retaining base ring, it is possible that not all of the binding elements are so attached, or even that the ring is not employed, where the binding elements or fabric substrate provides a sufficient shape-retaining quality to maintain the substantial concentricity of the windings.

Additionally, the primary coils described herein can also include a flexible magnetic shield as described in U.S. patent application Ser. No. 09/110,608, filed Jul. 6, 1998, entitled “Magnetic Shield for Primary Coil of Transcutaneous Energy Transfer Device”, and U.S. patent application Ser. No. 09/347,322, filed Jul. 2, 1999 entitled “Magnetic Shield for Primary Coil of Transcutaneous Energy Transfer Device”, which are hereby incorporated by reference in their entirety. The magnetic shield can cover the primary coil 114, 314, 414, 514, having a shape which is substantially the same as that of the primary coil, but the size of the shield would preferably be slightly larger that the primary coil.

An unshielded primary coil generates an alternating magnetic field which is directed in substantially equal parts in two directions: the first direction is toward the secondary coil; the second direction is away from the secondary coil. For several reasons, a shielded primary coil can be desirable. If the magnetic field directed away from the secondary coil can be diverted toward the secondary coil, the performance and efficiency of the TET could improve. Additionally, a conductive object in the vicinity of the shielded primary coil will be less likely to pass through the magnetic field of the coil and, therefore, will be less likely to cause variations in the energy transferred to the secondary coil. Since it is desired that this energy transfer be substantially uniform, the potential for spurious variations in energy transfer is at best undesirable, and at worst can have potentially adverse consequences for the patient.

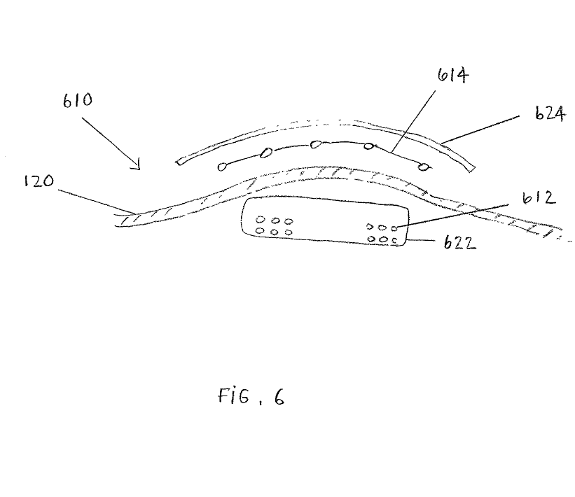

FIG. 6 illustrates a side cutaway view of an exemplary transcutaneous energy transfer system 600 comprising a TET coil pair 610 of the kind previously described, in combination with a magnetic shield 624. In use, the shield 624 can be placed on top of the flexible primary coil 614 of TET coil pair 610. The shield 624 can sit on the outer surface of the coil 614 which faces away from the patient. The shield 624 acts to divert the magnetic field that emanates from the primary coil 614 away from the exterior region external to the patient, causing the field intensity to increase in the region of secondary coil 612. This enhances the efficiency of energy transfer across the skin boundary 120 by the TET device 610 while decreasing the adverse effects of conductive elements that come into the vicinity of the primary coil.

In addition, the flexible magnetic shield 624 cooperatively functions with the primary coil 614 in conforming to the skin surface 120. To achieve this flexibility, the shield 624 can be formed of a low loss magnetic material in a flexible polymeric matrix, the shield 624 being formed of a ferrite powder in a silicon rubber for an illustrative embodiment. The shield 624 can, for example, include perforations to provide ventilation to the skin and to facilitate flexion of the shield 624.

In another embodiment, the shield 624 can be formed of a plurality of segments of a very high permeability but inflexible material, such as hard ferrite, which segments are connected to each other by a porous, flexible material. For example, the shield 624 can include a plurality of segments arranged in one or more nearly concentric rings, each concentric ring including segments of substantially the same size. The shield 624 can also be dimensioned and formed of a material that diverts a substantial portion of the magnetic field directed away from the exterior back toward the secondary coil 612.

The shield 624 and primary coil 614 can be mounted together to form a flexible primary coil assembly. A substantially water resistant coating can be applied to the assembly to make it substantially waterproof and easy to clean. In one particular embodiment, the primary coil assembly can be vinyl dip coated. Because the magnetic shield 624 is both flexible and porous, the primary coil assembly would be able to conform to the wearer's skin and allow for ventilation to the skin, in addition to being able to divert the magnetic field toward the secondary coil 612.

While the invention has been particularly shown and described above with reference to several preferred embodiments and variations thereon, it is to be understood that additional variations could be made in the invention by those skilled in the art while still remaining within the spirit and scope of the invention, and that the invention is intended to include any such variations, being limited only by the scope of the appended claims.

What is claimed is:

1. A flexible transcutaneous energy transfer primary coil, comprising:
a substantially shape-retaining base ring defining a primary plane;
a conducting element in the form of a plurality of substantially concentric windings attached to the base ring; and
at least one binding element, the binding element being bound to a plurality of the windings to substantially maintain the concentricity of the bound windings while allowing the bound windings to move in a direction transverse to the primary plane with respect to other bound windings, thereby enabling the primary coil to conform to a surface upon which the primary coil is applied.

2. The coil of claim 1, wherein the surface is a region of a patient's skin under which a secondary coil is implanted.

3. The coil of claim 2, wherein the conducting element is a flexible wire.

4. The coil of claim 3, wherein the wire is formed from Litzendraht wire.

5. The coil of claim 1, wherein the at least one binding element comprises a flexible substrate bound to each winding.

6. The coil of claim 5, wherein the flexible substrate is porous.

7. The coil of claim 5, wherein the flexible substrate is a fabric.

8. The coil of claim 7, wherein the fabric is spandex.

9. The coil of claim 7, wherein the fabric is a mesh fabric.

10. The coil of claim 7, wherein the flexible substrate is bound to the windings by stitches.

11. The coil of claim 1, wherein the at least one binding element comprises a plurality of threads.

12. The coil of claim 11, wherein each of the plurality of threads is bound to the base ring.

13. The coil of claim 11, wherein the threads are formed from an elastic material.

14. The coil of claim 5, wherein the flexible substrate is folded in an accordion fashion.

15. The coil of claim 1, further including a magnetic shield configured to cover at least a portion of the surface of the primary coil that faces away from the secondary coil when implanted in a patient.

16. The coil of claim 15, wherein the magnetic shield has substantially the same shape and a larger size than the primary coil.

17. The coil of claim 15, wherein the magnetic shield is flexible.

18. The coil of claim 17, wherein the magnetic shield is formed of a low loss magnetic material in a flexible polymeric matrix.

19. The coil of claim 18, wherein the magnetic material is ferrite, and the polymeric matrix is silicon rubber.

20. The coil of claim 17, wherein the magnetic shield includes perforations.

21. The coil of claim 17, wherein the magnetic shield is formed of a plurality of segments of a very high permeability material connected together by a porous, flexible material.

22. A coil for transferring electrical power to a subcutaneous utilization device, the coil being formed from a conducting element and having a series of windings, wherein the windings are attached to one another by at least one binding element, and a substantially shape-retaining base ring connected to the at least one binding element and surrounding the conducting element, wherein the at least one binding element and conducting element are constructed and arranged such that each winding is translatable to a location not in a plane that includes the base ring and non-translated windings.

23. The coil of claim 22, wherein the conducting element is a flexible wire.

24. The coil of claim 23, wherein the flexible wire is Litzendraht wire.

25. The coil of claim 22, wherein at least one the binding element comprises a flexible substrate bound to each winding.

26. The coil of claim 25, wherein the flexible substrate is porous.

27. The coil of claim 25, wherein the flexible substrate is a fabric.

28. The coil of claim 27, wherein the fabric is spandex.

29. The coil of claim 27, wherein the fabric is a mesh fabric.

30. The coil of claim 27, wherein the flexible substrate is bound to the windings by stitches.

31. The coil of claim 22, wherein the at least one binding element comprises a plurality of threads.

32. The coil of claim 31, wherein each of the plurality of threads is bound to the base ring and to at least one winding.

33. The coil of claim 31, wherein the threads are formed from elastic material.

34. The coil of claim 25, wherein the flexible substrate is folded in an accordion fashion.

35. The coil of claim 22, further including a magnetic shield configured to cover at least a portion of the surface of the primary coil that faces away from the secondary coil when implanted in a patient.

36. The coil of claim 35, wherein the magnetic shield has substantially the same shape and a larger size than the primary coil.

37. The coil of claim 35, wherein the magnetic shield is flexible.

38. The coil of claim 37, wherein the magnetic shield is formed of a low loss magnetic material in a flexible polymeric matrix.

39. The coil of claim 38, wherein the magnetic material is ferrite, and the polymeric matrix is silicon rubber.

40. The coil of claim 37, wherein the magnetic shield includes perforations.

41. The coil of claim 35, wherein the magnetic shield is formed of a plurality of segments of a very high permeability material connected together by a porous, flexible material.

42. A transcutaneous energy transfer system, comprising:
a flexible external primary coil to which energy to be transferred is applied; and
a secondary coil adapted to be coupled to the primary coil, the secondary coil being adapted to be connected to apply energy to an implanted utilization device;
the primary coil further comprising:
a substantially shape-retaining base ring defining a primary plane;
a conducting element in the form of a plurality of substantially concentric windings attached to the base ring; and
at least one binding element, the binding element being bound to a plurality of the windings to substantially maintain the concentricity of the bound windings while allowing the bound windings to move in a direction transverse to the primary plane with respect to other bound windings.

43. The system of claim 42, wherein the secondary coil is inductively coupled to the primary coil.

44. The system of claim 42, wherein when the primary coil is placed against the outer surface of the area where the secondary coil is implanted, the conducting element conforms to the shape of the outer surface.

45. The system of claim 44, wherein the conducting element comprises a flexible wire.

46. The system of claim 45, wherein the flexible wire is formed from Litzendraht wire.

47. The system of claim 42, wherein the at least one binding element comprises a flexible substrate bound to each winding.

48. The system of claim 47, wherein the flexible substrate is porous.

49. The system of claim 47, wherein the flexible substrate is a fabric.

50. The system of claim 42, wherein the at least one binding element comprises a plurality of threads.

51. The system of claim 50, wherein the threads are formed from an elastic material.

52. The system of claim 42, further including a magnetic shield configured to cover at least a portion of the surface of the primary coil that faces away from the secondary coil when implanted in a patient.

53. The system of claim 52, wherein the magnetic shield is flexible.

54. The system of claim 52, wherein the magnetic shield is porous.

55. An external primary coil for use in a transcutaneous energy transfer system having an implantable secondary coil configured to be inductively coupled to the external primary coil to deliver energy received from the primary coil to an implanted device, wherein the primary coil is flexible, taking on a shape in response to external forces such that when applied to a patient's skin under which the secondary coil is implanted, the primary coil continually conforms to the patient's skin over time.

56. A transcutaneous energy transfer system, comprising:
a flexible external primary coil to which energy to be transferred is applied; and
a secondary coil coupled to the primary coil, the secondary coil being implanted to be connected to apply energy to a subcutaneous utilization device;
the primary coil being formed from a conducting element and having a series of windings, each winding being able to flex with respect to an adjacent winding, wherein the windings are attached to one another by at least one binding element.

57. The system of claim 56, wherein the primary coil includes a substantially shape-retaining ring connected to the at least one binding element and surrounding the conducting element.

58. The system of claim 56, wherein the secondary coil is inductively coupled to the primary coil.

59. The system of claim 56, wherein when the primary coil is placed against the outer surface of the area where the secondary coil is implanted, the conducting element conforms to the shape of the outer surface.

60. The system of claim 59, wherein the conducting element is a flexible wire.

61. The system of claim 60, wherein the flexible wire is Litzendraht wire.

62. The system of claim 56, wherein the binding element comprises a flexible substrate bound to each winding.

63. The system of claim 62, wherein the flexible substrate is porous.

64. The system of claim 62, wherein the flexible substrate is a fabric.

65. The system of claim 56, wherein the binding element comprises a plurality of threads.

66. The system of claim 65, wherein the threads are formed from elastic material.

67. The system of claim 56, further including a magnetic shield configured to cover at least a portion of the surface of the primary coil that faces away from the secondary coil when implanted in a patient.

68. The system of claim 67, wherein the magnetic shield is flexible.

69. The system of claim 67, wherein the magnetic shield is porous.

70. An external primary coil for use in a transcutaneous energy transfer system having an implantable secondary coil configured to be inductively coupled to the external primary coil to deliver energy received from the primary coil to an implanted device, wherein individual windings of the primary coil can translate relative to each other to enable the primary coil to conform continually to a patient's skin under which the secondary coil is implanted.
